(19) United States
(12) Patent Application Publication
(10) Pub. No.: US 2020/0197430 A1
(43) Pub. Date: Jun. 25, 2020

(54) Combination Therapy

(71) Applicant: NuCana plc, Edinburgh (GB)

(72) Inventor: Hugh Griffith, Edinburgh (GB)

(21) Appl. No.: 16/642,832

(22) PCT Filed: Aug. 30, 2018
(86) PCT No.: PCT/GB2018/052445

§ 371 Date: Feb. 27, 2020

(30) Foreign Application Priority Data
Aug. 30, 2017 (GB) 1713914.8

(51) Int. Cl. A61K 31/7068; A61K 33/243; A61P 35/00

(52) U.S. Cl. CPC A61K 31/7068; A61P 35/00; A61K 33/243

(57) ABSTRACT

This invention relates to a combination of 2′-Deoxy-2′,2′-difluoro-D-cytidine-5′-O-[phenyl (benzoxy-L-alaninyl)] phosphate) (NUC-1031) and carboplatin, or other forms of platinum, and the use of the combination in treating cancer patients selected based on the patient's cancer's response to platinum. In particular the invention concerns the treatment of patients that have platinum sensitive cancers or platinum partially sensitive cancers.

This invention relates to a combination of 2′-Deoxy-2′,2′-difluoro-D-cytidine-5′-O-[phenyl (benzoxy- L-alaninyl)] phosphate) (NUC-1031) and carboplatin, or other forms of platinum, and the use of the combination in treating cancer patients selected based on the patient's cancer's response to platinum. In particular, to patients that have platinum sensitive cancers or platinum partially sensitive cancers.

### BACKGROUND

Gemcitabine (1; marketed as Gemzar®) is an effective nucleoside analogue that is currently approved to treat breast, non-small cell lung, ovarian and pancreatic cancers and widely used to treat a variety of other cancers including bladder, biliary, colorectal and lymphoma.

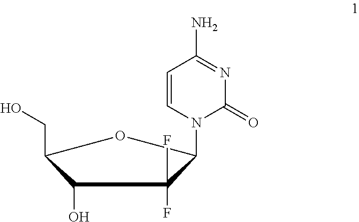

Gemcitabine's clinical utility is limited by a number of inherent and acquired resistance mechanisms. At the cellular level resistance is dependent on three parameters: (i) the down-regulation of deoxycytidine kinase, necessary for the activation into the phosphorylated moiety; (ii) the reduced expression of nucleoside transporters, in particular, hENT1, required for uptake by cancer cells; and (iii) the up-regulation of catalytic enzymes especially cytidine deaminase that degrades gemcitabine.

WO2005/012327 describes a series of nucleotide prodrugs for gemcitabine and related nucleoside drug molecules. Among them gemcitabine-[phenyl-benzoxy-L-alaninyl)]-phosphate (NUC-1031; chemical name: 2′-Deoxy-2′,2′-difluoro-D-cytidine-5′-O-[phenyl (benzoxy- L-alaninyl)] phosphate; 2) is identified as a particularly effective compound. These prodrugs appear to avoid many of the inherent and acquired resistance mechanisms which limit the utility of gemcitabine (Application of Pro Tide Technology to Gemcitabine: A Successful Approach to Overcome the Key Cancer Resistance Mechanisms Leads to a New Agent (NUC-1031) in Clinical Development'; Slusarczyk et all; J. Med. Chem.; 2014, 57, 1531-1542).

NUC-1031 2 is typically prepared as a mixture of two diastereoisomers, epimeric at the phosphate centre (the S-epimer 3 and the R-epimer 4) which can be separated and administered as a single epimer.

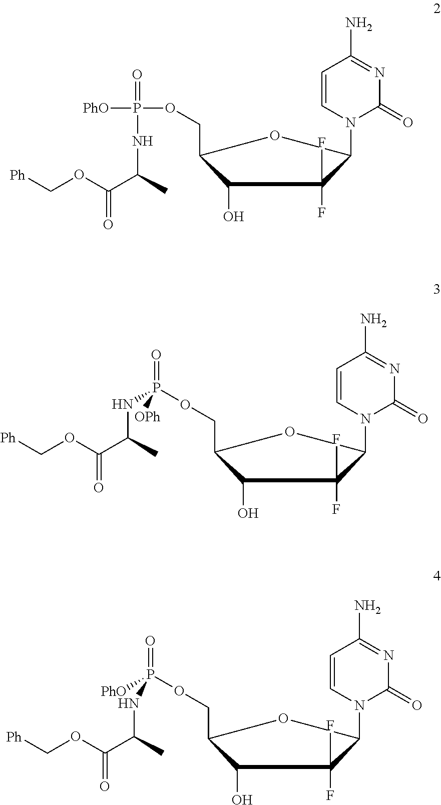

PRO-001 (aka ProGem1) was a first-in-human (FIH), phase I, open label, two stage study to investigate the safety, tolerability, clinical efficacy, pharmacokinetics (PK) and pharmacodynamics (PD) of NUC-1031 given in two parallel dosing schedules in subjects with advanced solid malignancies (EudraCT Number: 2011-005232-26). Subjects had the following tumour types at study entry: colorectal cancer (7 subjects), unknown primary (3), ovarian cancer (13), breast cancer (4), pancreatic cancer (9), cholangiocarcinoma (7), uterine or endometrial cancer (3), cervix cancer (2), lung cancer (6), mesothelioma (3) and thymus cancer (1). The study confirmed NUC-1031′s anti-tumour activity in patients with advanced progressive cancers, who have exhausted all standard therapeutic options, many of whom were resistant or refractory to prior nucleoside analogue therapy, including gemcitabine. Of particular note, the pharmacokinetic data showed that NUC-1031 as single agent generates around 217-fold higher Cₘₐₓ, intracellular levels of the active triphosphate moiety (dFdCTP) than reported for gemcitabine as a single agent at equimolar dose. Moreover, the analyses revealed that NUC-1031 releases lower levels of the potentially toxic metabolite, dFdU, normally associated with gemcitabine.

### Ovarian Cancer

Ovarian cancer is the sixth most commonly diagnosed cancer among women worldwide with an estimated 204,000 new cases diagnosed each year. The mortality rate is high with a 5 year survival rate of 45%, primarily because many women present with late stage disease but also because there is a high rate of disease recurrence and development of platinum-resistance. The initial therapy for advanced stage ovarian cancer is a combination of optimal surgical debulking and systemic treatment with a platinum plus taxane chemotherapy regimen. Even though complete response following treatment is observed in 70%, the majority will subsequently present with disease relapse within 2 years.

Therapy for recurrent disease is governed by the time between primary treatment with a platinum containing regimen and relapse. This is defined as the platinum-free interval (PFI): (1) Those with disease progression while receiving platinum-based therapy or within 4 weeks of last platinum dose are defined as having platinum-refractory disease; (2) those with a PFI of >1 month and less than 6 months have platinum-resistant disease; (3) those with a PFI of 6-12 months have partial platinum sensitivity; (4) and those with PFI>than 12 months have platinum-sensitive recurrence. According to NCl and NICE guidelines, those with platinum-resistant or refractory disease should then be treated with topotecan, liposomal doxorubicin, weekly paclitaxel or entered into a clinical study. Those with partially platinum-resistant recurrence should be treated with platinum-based regimens such as carboplatin with gemcitabine, carboplatin with liposomal doxorubicin or entered into a clinical study. Those with platinum-resistant disease should be retreated with carboplatin, either as monotherapy or in combination with paclitaxel. Unfortunately, those with platinum-resistant recurrence have a significantly shorter median overall survival (35 to 61 weeks) compared to those with platinum-sensitive disease (>104 weeks) suggesting that response to platinum is a key determinant for survival.

As used herein, platinum sensitive (PS) patients are those with a PFI>12 months, and platinum partially sensitive (PPS) patients are with a PFI>6 month and <12 months. Platinum resistant cancers are those that with a PFI of between 1 and 6 months; platinum refractory cancers progress while the patient is receiving platinum therapy or within one month of stopping a platinum containing therapy.

### Loss of Chemotherapy Sensitivity in Ovarian Cancer

An almost inevitable result of repeated exposure to courses of platinum-based chemotherapy is the development of a loss of platinum sensitivity and, eventually, platinum resistance or cancers become platinum refractory. This occurs via a number of mechanisms such as up-regulation of DNA damage repair and anti-apoptosis proteins, reduced copper transporters and increased drug efflux from the cancer cell. A rationale for combining platinum-based chemotherapy (such as carboplatin) with another chemotherapy agent, such as gemcitabine or liposomal doxorubicin, is to have the additive effect of two agents and to use the synergistic activity of gemcitabine to overcome resistance and improve sensitivity to platinum. Certainly, response to carboplatin alone in partially platinum resistant recurrence is inferior to carboplatin given in combination with gemcitabine or liposomal doxorubicin. Although the response to combination therapy is superior to platinum monotherapy, the overall prognosis for these patients is very poor and novel treatment strategies are needed for this population.

### Gemcitabine in Ovarian Cancer

Gemcitabine in combination with a platinum agent (e.g. carboplatin) is effective for relapsed platinum sensitive or partially sensitive ovarian cancer, even following previous platinum exposure, due to the synergistic action of gemcitabine with platinum and the ability of gemcitabine to overcome a degree of platinum resistance. In the AGO-OVAR study 356 patients with platinum sensitive recurrent ovarian cancer were assigned to either carboplatin AUC5 alone or carboplatin AUC4 (day 1) plus gemcitabine 1000 mg/m² on days 1 & 8 every 3 weeks. After a median follow-up of 17 months, a median PFS of 8.6 months was observed (95% Cl, 7.9 to 9.7 months) for gemcitabine plus carboplatin and 5.8 months (95% Cl, 5.2 to 7.1 months) for carboplatin alone (HR 0.72 (95% Cl, 0.58 to 0.90; P=0.0031)). A response rate of 47.2% (95% Cl, 39.9% to 54.5%) was recorded for gemcitabine plus carboplatin and 30.9% (95% Cl, 24.1% to 37.7%) for carboplatin alone (P=0.0016). In view of the improved response and survival, gemcitabine is usually given alongside carboplatin in the relapsed, platinum-sensitive setting.

The combination of carboplatin AUC4 (day 1) and gemcitabine 1000 mg/m² on (days 1 & 8) every 3 weeks was trialled in the control arm of the OCEANS study, a randomised study in patients with platinum-sensitive ovarian cancer. 242 patients received this combination and demonstrated an objective response rate of 57.4%, with 48.3% having a partial response, and a duration of response of 7.4 months. In women with platinum-resistant recurrence, gemcitabine given alongside carboplatin was shown in one study to have a global response rate of 47%.

Combination of NUC-1031 and carboplatin

A Phase IB open label, dose-escalation study, PRO-002 has been conducted on the combination of NUC-1031 and carboplatin. Some results are disclosed in WO2017/060661.

It is an aim of this invention to provide a combination therapy for treating cancer. It is an aim of this invention to provide a therapy that is more effective than existing treatments.

Certain embodiments of this invention satisfy some or all of the above aims.

### BRIEF SUMMARY OF THE DISCLOSURE

In accordance with the present invention there is provided NUC-1031, or a pharmaceutically acceptable salt or solvate thereof for use in a method of treating cancer in combination with a platinum agent, wherein the method comprises determining the platinum status of the patient and administering the NUC-1031 and platinum agent to a patient identified as likely to respond to treatment based on their platinum status. In one embodiment, the treatment stage of the patient (e.g. the number of previous treatment regimens the patient has received) is also taken into consideration. In particular embodiments, the patient is selected for treatment with NUC-1031/platinum if (i) the patient's cancer is platinum sensitive or partially sensitive and the patient has received at least one prior treatment regimen; (ii) the patient's cancer is platinum resistant and the patient has received at least two prior treatment regimens; or (iii) the patient's cancer is platinum refractory and the patient has received at least three prior treatment regimens.

In accordance with the present invention there is also provided NUC-1031, or a pharmaceutically acceptable salt or solvate thereof for use in a method of treating cancer in combination with platinum agent, wherein the cancer is a platinum sensitive cancer or a platinum partially sensitive cancer. In one embodiment, the method of treatment involves determining the platinum status of the patient's cancer, optionally making a treatment decision based on this status in conjunction with the number of previous treatments the patient has received, and then administering to the patient pharmaceutically effective amounts of NUC-1031 and a platinum agent, such as carboplatin or cisplatin. In one embodiment, the platinum agent is carboplatin which is administered at a dose in the range of AUC 4 to 6 and the NUC-1031 is administered at a dose in the range from 350 to 750 mg/m².

As used herein, suitable platinum agents include: carboplatin, cisplatin, oxaliplatin and nedaplatin.

In accordance with the present invention there is provided NUC-1031, or a pharmaceutically acceptable salt or solvate thereof for use in a method of treating cancer in combination with a platinum agent, wherein the NUC-1031 is administered at a dose in the range from 350 to 750 mg/m². In one embodiment, the method of treatment involves determining the platinum status of the patient's cancer, optionally making a treatment decision based on this status in conjunction with the number of previous treatments the patient has received, and then administering to the patient pharmaceutically effective amounts of NUC-1031 and a platinum agent, such as carboplatin or cisplatin. In one embodiment, the platinum agent is carboplatin which is administered at a dose in the range of AUC 4 to 6.

In accordance with the present invention there is provided NUC-1031, or a pharmaceutically acceptable salt or solvate thereof for use in treating cancer in combination with carboplatin, wherein the NUC-1031 is administered at a dose in the range from 350 to 750 mg/m² and the carboplatin is administered at a dose in the range from AUC 4 to 6, such as AUC 4.5 to AUC 6.

In one embodiment, the cancer is a platinum sensitive cancer or a platinum partially sensitive cancer. In particular embodiments, the NUC-1031 is administered at a starting dose in the range from 350 to 750 mg/m² and the carboplatin is administered at a starting dose in the range from AUC 4 to AUC 6, and the cancer is a platinum sensitive cancer or a platinum partially sensitive cancer.

In other embodiments, the cancer is a platinum resistant or refractory cancer. In particular embodiments, the NUC-1031 is administered at a starting dose in the range from 350 to 750 mg/m² and the carboplatin is administered at a starting dose in the range from AUC 4 to AUC 6.

The invention also provides a method of treating cancer, the method comprising administering to a subject in need thereof a therapeutically effective amount of NUC-1031, or a pharmaceutically acceptable salt or solvate thereof, in combination with carboplatin, wherein the NUC-1031 is administered at a dose in the range from 350 to 750 mg/m² and the carboplatin is administered at a dose in the range from AUC 4 to AUC 6, such as AUC 4.5 to 6, and the cancer is a platinum sensitive cancer or a platinum partially sensitive cancer.

The invention also provides a method of treating cancer, the method comprising determining the platinum status of the patient's cancer, optionally making a treatment decision based on this status in conjunction with the number of previous treatments the patient has received, and administering to a subject in need thereof a therapeutically effective amount of NUC-1031, or a pharmaceutically acceptable salt or solvate thereof, in combination with a platinum agent, wherein the NUC-1031 is administered at a dose in the range from 350 to 750 mg/m² In one embodiment, the platinum agent is carboplatin which is administered at a dose in the range from AUC 4 to AUC 6, such as AUC 4.5 to 6. In another embodiment, the cancer is a platinum sensitive cancer or a platinum partially sensitive cancer.

In one embodiment, the method of treatment involves determining the susceptibility of the patient's cancer to platinum treatment and making the treatment decision (e.g. whether to administer the NUC-1031/platinum agent combination or not) based on this platinum sensitivity determination. In a particular embodiment, if the patient's cancer is a platinum sensitive cancer or a platinum partially sensitive cancer the patient is administered the NUC-1031/platinum agent (or salts thereof) combination in the doses recited above. As noted above, a patient may be selected for treatment (and/or treated) with NUC-1031/platinum if: (i) the patient's cancer is platinum sensitive or partially sensitive and the patient has received at least one prior treatment regimen (such as 1, 2,3,4, 5, 6 or more prior treatments); (ii) the patient's cancer is platinum resistant and the patient has received at least two (such as 2, 3, 4, 5, 6 or more) prior treatment regimens; or (iii) the patient's cancer is platinum refractory and the patient has received at least three (such as 3, 4, 5, 6 or more prior treatments) prior treatment regimens.

The invention also provides NUC-1031, or a pharmaceutically acceptable salt or solvate thereof, for use in a method of treating a patient with cancer in combination with a platinum agent, wherein the method involves determining the sensitivity of the patient's cancer to platinum treatment and if the patient's cancer is determined to be a platinum sensitive cancer or a platinum partially sensitive cancer administering to the patient the NUC-1031 at a dose in the range from 350 to 750 mg/m². In one embodiment, the patient has undergone at least one prior therapy (treatment regime). In a particular embodiment, the patient has undergone platinum therapy with an agent such as carboplatin, cisplatin, oxaliplatin and nedaplatin.

The invention also provides NUC-1031, or a pharmaceutically acceptable salt or solvate thereof, for use in a method of treating a patient with cancer in combination with carboplatin, wherein the method involves determining the sensitivity of the patient's cancer to platinum treatment and if the patient's cancer is determined to be a platinum sensitive cancer or a platinum partially sensitive cancer administering to the patient the NUC-1031 at a starting dose in the range from 350 to 750 mg/m² and the carboplatin at a dose in the range from AUC 4 to AUC 6, such as AUC 4.5 to 6.

The invention also provides NUC-1031, or a pharmaceutically acceptable salt or solvate thereof, for use in the manufacture of a medicament for treating cancer in combination with carboplatin, wherein the NUC-1031 may be for administration at a dose in the range from 350 to 750 mg/m² and the carboplatin is administered at a dose in the range from AUC 4 to AUC 6, such as AUC 4.5 to 6, and the cancer is a platinum sensitive cancer or a platinum partially sensitive cancer.

The invention also provides NUC-1031, or a pharmaceutically acceptable salt or solvate thereof, for use in a method of treating a patient with cancer in combination with a platinum agent, such as carboplatin or cisplatin, wherein the patient has received at least one previous treatment and the cancer is platinum sensitive or partially sensitive. In particular embodiments, the patient with platinum sensitive or platinum partially sensitive cancer has received at least two prior treatments (treatment regimens).

The invention also provides NUC-1031, or a pharmaceutically acceptable salt or solvate thereof, for use in a method of treating a patient with cancer in combination with a platinum agent, such as carboplatin or cisplatin, wherein the patient has received at least two prior treatments and the cancer is platinum resistant. In particular embodiments, the patient with platinum resistant cancer has received at least three prior treatments (treatment regimens).

The invention also provides NUC-1031, or a pharmaceutically acceptable salt or solvate thereof, for use in a method of treating a patient with cancer in combination with a platinum agent, such as carboplatin or cisplatin, wherein the patient has received at least three prior treatments and the cancer is platinum refractory.

### DETAILED DISCLOSURE

The present invention is based upon the inventors' identification of a dosing combination of NUC-1031 and carboplatin that offers particular advantages in the treatment of patients with particular platinum cancer statuses, such as platinum sensitive (PS) or platinum partially sensitive (PPS) cancers. While the combination of NUC-1031 and carboplatin has previously been considered in the treatment of cancer (WO2017/060661), the particular type of patients (characterised with reference to the nature of their cancer and making a treatment decision based on their platinum response status, optionally in conjunction with the number of prior treatment regimens the patient has received) and doses of the active agents identified herein has not previously been disclosed.

The inventors have found that the most effective treatment response is found in patients whose cancer is a platinum sensitive cancer or a platinum partially sensitive cancer and thus, that the platinum response status can be used to select patients for treatment with the combination of NUC-1031 and carboplatin at the doses recited herein.

The inventors have also found effective treatment responses in patients whose cancer is platinum resistant and who have undergone at least two previous treatment regimens, and thus that the platinum response status can be used to select patients with platinum resistant cancer for third line or further line treatment with the combination of NUC-1031 and carboplatin at the doses recited herein.

The inventors have also found effective treatment responses in patients whose cancer is platinum refractory and who have undergone at least three previous treatment regimens, and thus that the platinum response status can be used to select patients with platinum refractory cancer for fourth line or further line treatment with the combination of NUC-1031 and carboplatin at the doses recited herein.

### Platinum Status

Platinum status refers to the response that the cancer has to platinum therapy. The sensitivity of the patient's cancer to platinum treatment is used as the basis for determining to treat with the NUC-1031 and the platinum agent, such as carboplatin or cisplatin. The number of previous treatment regimens/regimes that the patient has received may be used in conjunction with the platinum status in order to determine the basis for determining to treat with the NUC-1031 and the platinum agent, such as carboplatin or cisplatin.

The platinum status of the patient's cancer can be determined directly based on their response to treatment with a platinum drug, or it may be determined based on measurement of a surrogate marker of platinum status.

### Platinum Agent

The results observed with the combination of NUC-1031 and carboplatin are predicted to be seen when combining NUC-1031 with any platinum agent, such as cisplatin, oxaliplatin and nedaplatin.

### Dosages

The inventors have found that the combination of the selected doses and selected patient group confers advantages in terms of both the clinical outcome of treatment, and also the reduction of unwanted side effects.

Remarkably, the inventors have found that the combination of approximately 350 to 750 mg/m² NUC-1031, such as 350 to 550 mg/m², and carboplatin at approximately AUC 4 to 6, such as AUC 4.5 to 6, lead to significantly improved outcomes in patients with PS or PPS cancer when compared to the combination of gemcitabine and carboplatin. These improvements are noted when compared to either treatment using the same agents and doses in populations of patients that are platinum resistant, or have not been categorised with reference to their platinum sensitivity status, or when compared to PS or PPS patients receiving gemcitabine (the parent compound from which NUC-1031 is derived) and carboplatin, a combination that is the standard of care for cancers of this sort. Indeed, significant improvements are also seen in patients whose cancer was platinum resistant or refractory. As can be seen from Column 2 in Table 1, fourth line patients that were resistant to platinum displayed a PFS of 7.4 months and had far fewer toxic side effects than even second line patients treated with gemcitabine and carboplatin. NUC-1031 appears to help re-sensitise the patient's cancer to platinum treatment. The NUC-1031 then also acts in a synergistic manner with carboplatin to increase cancer cell killing and reduce off-target toxic side effects.

It is noteworthy also that the use of NUC-1031 in place of gemcitabine allows the use of carboplatin at a higher AUC (4 to 6 rather than Carboplatin can be used at AUCS when administered as a monotherapy, but due to toxicity must be used at AUC when in combination with gemcitabine. NUC-1031 however, can be used in combination with carboplatin at AUC>5. This means that an optimal dose of carboplatin can be used when combined with NUC-1031, and that this combination is likely to have a more favourable safety profile in treated patients.

As set out in more detail in the Examples, the overall response rate for patients receiving treatment in accordance with the medical uses or methods of the invention was 67%, as compared to an overall response rate of 47% in those patients receiving gemcitabine and carboplatin. It should be noted that in the Example disclosed herein, the patients receiving gem/carbo were less heavily pre-treated (i.e. second line only) than the patients receiving NUC-1031/carboplatin (third and fourth line; who would therefore have been expected to have a better objective response rate). Indeed, as a rule of thumb it is predicted that PFS duration halves for every additional treatment regimen the patient has received. Thus, for example, a PFS of 6 months in second line treatment would be expected to be about 3 months in third line treatment. Furthermore, whilst the gemcitabine/carboplatin worked favourably in the PS population, little difference was seen in the PPS population. It thus appears that NUC-1031/carboplatin has enhanced effects in one-line of therapy later (those with a poorer prognosis generally) and a poorer Pt status.

Furthermore, the progression free survival (PFS) period for PS or PPS patients receiving treatment in accordance with the medical uses or methods of treatment of the invention was improved as compared to controls. Median PFS for PS or PPS patients treated in accordance with the invention was 8.6 months, while PS or PPS patients receiving gemcitabine and carboplatin also had PFS of 8.6 months (but in patients treated as second line), these patients suffered from significantly more side effects (such as neutropenia and thrombocytopenia; see Table 1). The difference between the PFS for PS and PPS patients receiving treatment in accordance with the invention, and for unsorted or platinum resistant patients was even more pronounced (9.4 months vs 7.4 months).

Not only was clinical outcome in patients treated in accordance with the invention much better than in controls, but the side effects observed using the medical uses of methods of treatment of the invention were surprisingly reduced. PS or PPS patients receiving treatment in accordance with the invention exhibited no thrombocytopenia, as compared to reported rates of 35% in the standard of care group, and 53% and 52% respectively in the platinum resistant and unsorted patients receiving 500 mg/m² NUC-1031 and carboplatin AUCS. Neutropenia rates were lower than, but comparable to, the platinum resistant and unsorted patients receiving the same treatments, and 21% lower (50% as compared to 71%) as compared to gem/carbo.

It is important to note that the remarkable results achieved by the medical uses and methods of treatment of the invention are available in subjects who have advanced cancers that have already proved unresponsive to treatment. As set out in the Examples, the advantages noted occurred in patients who were receiving the medical uses or methods of treatment of the invention after an average of three lines of cancer therapy. It would not be expected that such heavily pre-treated populations, with advanced and intractable cancers, would respond as positively as they have been found to do.

The treatments of the present invention are based on the fact that the combination of the two agents (i.e. the gemcitabine-[phenyl-benzoxy-L-alaninyl)]-phosphate and the carboplatin) show greater efficacy when administered in combination at a specific dosage range than is the case when either is administered alone. The term ‘in combination’ or ‘together’ in the context of the present invention refers to the fact that the two agents are both administered to the same patient during the treatment period. The administration may be separate in the sense of being provided in separate doses or may be in the same dose. Administration may take place concurrently or in sequence either immediately one after the other or with a time interval in between the administration of the two agents. The term ‘alone’ in the context of this discussion thus means administration of only one active agent and no administration of the other agent during the treatment period, even after a time interval.

Combination therapy according to the invention embraces the co-administration or sequential administration of the two active agents in a manner which enhances the overall therapeutic result relative to the administration of one of the active agents alone during the overall treatment period. The pharmaceutical formulation(s) employed for the purpose may be individual, i.e. separate formulations, or presented in a single formulation. The formulation or each formulation may be in a liquid form, either diluted or ready for dilution, or may be in a solid form. Solid forms may be provided for dissolution in a suitable solvent medium. Solid forms may also be presented in concentrated unit dosage form as tablets, capsules losanges etc.

The NUC-1031 may be a mixture of phosphate diastereoisomers or it may be the (S)-epimer or as the (R)-epimer in substantially diastereomerically pure form. It may be that the NUC-1031 is NUC-1031 in substantially diastereomerically pure form. ‘Substantially diastereomerically pure’ is defined for the purposes of this invention as a diastereomeric purity of greater than about 90%. If present as a substantially diastereoisomerically pure form, the NUC-1031 may have a diastereoisomeric purity of greater than 95%, 98%, 99%, or even 99.5%.

### Cancer

The cancer may be a cancer selected from but not restricted to: pancreatic cancer, breast cancer, ovarian cancer, bladder cancer, colorectal cancer, lung cancer, billiary tract cancer, prostate cancer, renal cancer, lymphoma, leukaemia, cervical cancer, thymic cancer, glioblastoma multiforme, a cancer of an unknown primary origin, oesophageal cancer, mesothelioma, adrenal cancer, cancer of the uterus, cancer of the fallopian tube, peritoneal cancer, endometrial cancer, testicular cancer, head and neck cancer, cancer of the central nervous system and germ cell tumours.

The cancer may be selected from, but not restricted to pancreatic cancer, lung cancer, bladder cancer, breast cancer, biliary cancer, colorectal cancer and a gynaecological cancer. The cancer may be of gynaecological origin (e.g. a cancer of the uterus, cancer of the fallopian tube, cancer of the endometrium, cancer of the ovary, cancer of the peritoneum and cancer of the cervix). The cancer may be selected from ovarian cancer, fallopian tube cancer; and peritoneal cancer. Suitably the ovarian cancer may be epithelial ovarian cancer. Suitably the peritoneal cancer may be primary peritoneal cancer. The inventors have found that the medical uses and methods of treatment of the invention are particularly beneficial in the treatment of cancers selected from the group consisting of: ovarian cancer, fallopian tube cancer; and peritoneal cancer.

The inventors have found that the medical uses and methods of treatment of the invention are particularly beneficial in the treatment of PS or PPS cancers selected from the group consisting of: ovarian cancer, fallopian tube cancer; and peritoneal cancer.

Similar to gemcitabine, NUC-1031 seems to re-sensitise patients to platinum treatment. Thus, patients that have become platinum resistant regain platinum sensitivity.

The cancer may be a platinum sensitive cancer. The cancer may be a platinum partially sensitive cancer. The cancer may be a platinum resistant cancer. The cancer may be a platinum refractory cancer.

The cancer patient may have received one prior treatment regime. The said prior treatment regime may have been treatment with a platinum agent. The cancer patient may have received at least two prior treatment regimes. One of said prior treatment regime may have been treatment with a platinum agent. The cancer patient may have received at least three prior treatment regimes. One of said prior treatment regime may have been treatment with a platinum agent.

The cancer may be a platinum sensitive or platinum partially sensitive cancer and the cancer patient may have received one prior treatment regime.

The cancer may be a platinum resistant cancer and the cancer patient may have received at least two prior treatment regimes.

The cancer may be a platinum refractory cancer and the cancer patient may have received at least three prior treatment regimes.

### Patient Groups

The patient may have received one or more previous treatments (lines of treatment). A patient that is particularly suited to the treatments as described herein include: (i) a patient that has received at least one previous treatment and is platinum sensitive or partially sensitive; (ii) a patient that has received at least two prior treatment and is platinum resistant; or (iii) a patient that has received at least three prior treatments and is platinum refractory.

The patient may be selected for NUC-1031/platinum combination treatment according to the invention described herein, based on their platinum response status. For example, a putative cancer patient is assessed to determine whether their cancer is platinum-refractory, platinum-resistant, platinum sensitive or platinum partially sensitive cancer. For example, if the patient's cancer disease is classified as platinum sensitive or platinum partially sensitive the patient is suitable for and/or positively selected for treatment with the NUC-1031/platinum combination as described herein, particularly if the patient has received at least one prior treatment regimen. In this way, the platinum response status (e.g. platinum-refractory, platinum-resistant, platinum sensitive or platinum partially sensitive) is used to decide the treatment for the patient and is thus part of the method of treatment of a cancer patient.

The cancer may be relapsed. The cancer may be previously untreated. Suitably, treatment in accordance with the medical uses or methods of the invention may be provided as a first line cancer therapy (i.e. the first cancer therapy provided after diagnosis of the disease). Alternatively, it may be used as a second or further line cancer treatment. It may be used as a third or further line cancer treatment. It may be used as a fourth or further line cancer treatment.

### Compounds

The compounds of the invention may be obtained, stored and/or administered in the form of a pharmaceutically acceptable salt. Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate, hemioxalate and hemicalcium salts. In certain embodiments, particularly those that apply to the s-epimer, the compound is in the form of a HCl salt or a hemioxalate salt.

A solvate will typically be a hydrate. Thus, the NUC-1031 may be in the form of a salt or hydrate, or a solvate (e.g. hydrate) of a salt). The NUC-1031 may be in the form of a solvate, e.g. a cocrystal. Illustrative coformers for cocrystals include: oxalic acid, maleic acid, malonic acid, glutaric acid, succinic acid, sulfamethoxypyridazine, benzoic acid, 4,4′-dipyridyl, nicotinamide, and saccharin.

It may be that the NUC-1031 is not in the form of a salt and it may be that it is not in the form of a solvate or hydrate. Preferably, the NUC-1031 is in the form of the free base.

The NUC-1031 and the carboplatin may be administered simultaneously, or they may be administered sequentially. Where they are administered simultaneously, they may be administered in a single formulation or they may be administered in separate formulations. Where they are administered sequentially, they may be administered on the same day or they may be administered on separate days during the treatment period. It may be that on certain days during the treatment period, the NUC-1031 and the carboplatin are administered simultaneously or on the same day and on certain other days in the treatment program a single one of the agents is administered.

### NUC-1031 Formulations

The NUC-1031 may be administered parenterally, e.g. for intravenously, subcutaneously or intramuscularly. Preferably, the NUC-1031 is administered intravenously.

The NUC-1031 may be administered parenterally as an aqueous formulation which optionally also comprises a polar organic solvent, e.g. dimethylacetamide (DMA) together with a surfactant

The formulation may be for dilution by a predetermined amount shortly before administration, i.e. up to 48 hours (e.g. up to 24, 12 or 2 hours) before administration.

The formulation may also comprise one or more pharmaceutically acceptable solubilizers, e.g. a pharmaceutically acceptable non-ionic solubilizers. Solubilizers may also be called surfactants or emulsifiers. Illustrative solubilizers include polyethoxylated fatty acids and fatty acid esters and mixtures thereof. Suitable solubilizers may be or may comprise polyethoxylated castor oil (e.g. that sold under the trade name Kolliphor® ELP); or may be or may comprise polyethoxylated hydroxy-stearic acid (e.g. that sold under the trade names Solutol® or Kolliphor® HS15); or may be or comprise polyethoxylated (e.g. polyoxyethylene (20)) sorbitan monooleate, (e.g. that sold under the trade name Tween® 80).

In certain preferred embodiments, the formulation comprises more than one pharmaceutically acceptable solubilizer.

The formulation may also comprise an aqueous vehicle. The formulation may be ready to administer, in which case it will typically comprise an aqueous vehicle.

The formulation may be for parenteral, e.g. for intravenous, subcutaneous or intramuscular administration. Preferably, the formulation is for intravenous administration. The administration may be through a central vein or it may be through a peripheral vein.

The formulation may be a formulation described in WO2015/198059.

While NUC-1031 is preferably formulated for parenteral administration, in certain embodiments of the invention it may be administered orally.

Any of the above-mentioned formulations may also comprise the carboplatin.

Formulations of carboplatin

Carboplatin may be administered parenterally, e.g. intravenously, intraperitoneally, subcutaneously or intramuscularly. Preferably, carboplatin is administered intravenously.

Carboplatin will typically be administered as an aqueous solution, e.g. as a sterile, pyrogen-free, 10 mg/mL aqueous solution. Further information on the administration of carboplatin is available, for example, on the US FDA approved label for Paraplatin®.

Carboplatin is typically administered by infusion into a vein but it may be administered intraperitoneally. Where carboplatin is administered intravenously, this may be done over 15 to 60 minutes or it may be over a longer period, e.g. continuous IV infusion over 24 hours.

Formulations of cisplatin

Cisplatin may be administered parenterally, e.g. intravenously, intraperitoneally, subcutaneously or intramuscularly. Preferably, carboplatin is administered intravenously.

Cisplatin will typically be administered as an aqueous solution, e.g. as a sterile, pyrogen-free, 10 mg/mL aqueous solution. Further information on the administration of carboplatin is available, for example, on the US FDA approved label for Platinol®.

Cisplatin is typically administered by infusion into a vein but it may be administered intraperitoneally. Where carboplatin is administered intravenously, this may be done over 15 to 60 minutes or it may be over a longer period, e.g. continuous IV infusion over 24 hours.

### Dosage Regimens

It may be that the NUC-1031 is administered at a dose in the range from 400 mg/m² to 600 mg/m². It may be that the NUC-1031 is administered at a dose in the range from 450 mg/m² to 550 mg/m². It may be that the NUC-1031 is administered at a dose of approximately 500 mg/m². It may be that the carboplatin is administered at a dose in the range from AUC 4 to 6, such as AUC 4.5 to AUC 5.5. It may be that the carboplatin is administered at a dose in the range from AUC 4.75 to AUC 5.25. It may be that the carboplatin is administered at a dose of approximately AUC 5. It may be that the NUC-1031 is administered at a dose of approximately 500 mg/m² and the carboplatin is administered at a dose of approximately AUC 5.

It may be that the NUC-1031 is administered twice in a 21 day cycle (dosing schedule or program). It may be that platinum agent (such as carboplatin, cisplatin, oxaliplatin or nedaplatin) is administered once or twice in the 21 day cycle. In a particular dosage regimen NUC-1031 is administered on day 1 and day 8 of a 21 day cycle and platinum agent is administered on day 1 of the 21 day cycle. It may be that NUC-1031 and the platinum agent are administered simultaneously on day 1 of a 21 day cycle.

It may be that the dose of NUC-1031, or the dose of the platinum agent (such as carboplatin, cisplatin, oxaliplatin or nedaplatin), or the dose of both of the compounds, decreases from the first treatment cycle to the second (or subsequent) treatment cycle. Preferably, however, the dose of NUC-1031 and the dose of the platinum agent remains substantially the same in each treatment cycle.

It may be that the dose of NUC-1031, or the dose of carboplatin, or the dose of both of the compounds, decreases from the first treatment cycle (starting dose) to the second (or subsequent) treatment cycle. Preferably, however, the dose of NUC-1031 and the dose of carboplatin remains substantially the same in each treatment cycle.

When used in combination with NUC-1031 cisplatin is administered at a dose in the range from 10 to 100 mg/m²/day, such as 20-40 mg/m²/day. It may be that the cisplatin is administered at a dose of 25, 30 or 45 mg/m²/day.

It may be that the NUC-1031 is administered at a dose of approximately 500 mg/m² and the cisplatin is administered at a dose of approximately 25 mg/m²/day.

It may be that the dose of NUC-1031, or the dose of the cisplatin, or the dose of both of the compounds, decreases from the first treatment cycle to the second (or subsequent) treatment cycle. Preferably, however, the dose of NUC-1031 and the dose of carboplatin remains substantially the same in each treatment cycle.

It may be that the NUC-1031 is administered twice in a 21 day cycle. In a particular dosage regimen NUC-1031 and the cisplatin are both administered on day 1 and day 8 of a 21 day cycle.

The person of skill in the art is able to determine suitable formulations and dosage regimes for platinum agents other than carboplatin or cisplatin.

It may be that the above-mentioned dosage regimen provides an improved survival rate in patients. It may be that it provides a stable disease in greater than 30%, such as greater than 40%, 50% or greater than 60% of patients. It may be that it provides a reduction of CA125 of greater than 30%, such as greater than 40%, greater than 50% or greater than 60% in greater than 50% of patients. It may be that it provides one or more of the above benefits with an acceptable level of side-effects. It may be that it provides one or more of the above benefits with a reduced incidence of thrombocytopenia compared to the incidence seen with gemcitabine and the platinum agent (e.g. carboplatin, cisplatin, oxaliplatin or nedaplatin). The incidence of thrombocytopenia in PS and/or PPS patient groups may be, for example, less than 40%, less than 30%, less than 20%, less than 10%, less than 5%, less than 2% or less than 1%. It may be that it provides one or more of the above benefits with a reduced incidence of neutropenia compared to the incidence seen with gemcitabine and carboplatin. The incidence of neutropenia in PS and/or PPS patient groups may be, for example, less than 65%, less than 60%, less than 55%, less than 50%, less than 44% or less than 30%.

The incidence of thrombocytopenia in Pt resistant and/or Pt refractory patient groups may be, for example, less than 50%, less than 45%, less than 40%, less than 30%, less than 20%, less than 10%, less than 5%, less than 2% or less than 1%. It may be that it provides one or more of the above benefits with a reduced incidence of neutropenia compared to the incidence seen with gemcitabine and carboplatin. The incidence of neutropenia in Pt resistant and/or Pt refractory patient groups may be, for example, less than 60%, less than 55%, less than 50%, less than 44% or less than 30%.

The medical uses or methods of treatment of the invention may be utilised for as many cycles of treatment as are considered to be clinically relevant. For example, treatment may be provided to a subject in need thereof for at least 5 cycles of treatment, at least 6 cycles of treatment, at least 7 cycles of treatment, at least 8 cycles of treatment, at least 9 cycles of treatment, or at least 10 cycles of treatment.

### DETAILED DESCRIPTION

‘Simultaneous’ is intended to mean “substantially simultaneous” e.g. less than 30 mins apart. ‘Sequential’ means administration more than 30 mins apart.

Throughout this specification, the term ‘a compound of the invention’ is intended to mean gemcitabine-[phenyl-benzoxy-L-alaninyl)]-phosphate. It may be in the form of the S-epimer, the R-epimer or a mixture therefore. Thus, the compound of the invention may be a compound of formula 2, 3 or 4, or mixtures of 3 and 4.

Throughout this specification, the term S-epimer or S-diastereoisomer refers to gemcitabine-[phenyl-benzoxy-L-alaninyl)]-(S)-phosphate. Likewise, throughout this specification, the term R-epimer or R-diastereoisomer refers to gemcitabine-[phenyl-benzoxy-L-alaninyl)]-(R)-phosphate.

The compounds of the invention may be obtained, stored and/or administered in the form of a pharmaceutically acceptable salt. Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate, hemioxalate and hemicalcium salts.

Compounds of the invention may exist in a single crystal form or in a mixture of crystal forms or they may be amorphous. Thus, compounds of the invention intended for pharmaceutical use may be administered as crystalline or amorphous products. They may be obtained, for example, as solid plugs, powders, or films by methods such as precipitation, crystallization, freeze drying, or spray drying, or evaporative drying. Microwave or radio frequency drying may be used for this purpose.

The appropriate dose to obtain a desired AUC of carboplatin may be calculated using the Calvert formula (dose in mg=AUC [glomerular filtration rate+25] (Calvert et al., J. Clin. Oncol. 7:1748-1756, 1989), which takes into consideration factors such as sex, age, weight, and renal clearance rate (glomerular filtration rate). AUC 5 means a dose of 5 mg/mL×min. Maximum carboplatin dose (mg)=target AUC (mg×min/mL)×(150 ml/min); so, the maximum AUC5 dose is 5×150=750 mg.

A compound of the invention, or pharmaceutically acceptable salt or solvate thereof, may be used on their own but will generally be administered in the form of a pharmaceutical composition in which the compounds of the invention, or pharmaceutically acceptable salt or solvate thereof, is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration of the compounds of the invention, the pharmaceutical composition which is used to administer the compounds of the invention will preferably comprise from 0.05 to 99%w (per cent by weight) compounds of the invention, more preferably from 0.05 to 80%w compounds of the invention, still more preferably from 0.10 to 70%w compounds of the invention, and even more preferably from 0.10 to 50%w compounds of the invention, all percentages by weight being based on total composition.

For oral administration the compounds of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compounds of the invention may be admixed with, for example, a vegetable oil or polyethylene glycol. Hard gelatine capsules may contain granules of the compound using either the above-mentioned excipients for tablets.

Also, liquid or semisolid formulations of the compound of the invention may be filled into hard gelatine capsules.

Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, sweetening agents (such as saccharine), preservative agents and/or carboxymethylcellulose as a thickening agent or other excipients known to those skilled in art.

For parenteral (e.g. intravenous) administration the compounds may be administered as a sterile aqueous or oily solution. The compounds of the invention are very lipophillic.

Aqueous formulations will typically, therefore, also contain a pharmaceutically acceptable polar organic solvent.

The present invention also includes all pharmaceutically acceptable isotopically-labelled forms of compounds 2, 3 or 4 wherein one or more atoms are replaced by atoms having the same atomic number, but an atomic mass or mass number different from the atomic mass or mass number of the predominant isotope usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labelled compounds can generally be prepared by conventional techniques known to those skilled in the art or by processes analogous to those described using an appropriate isotopically-labelled reagent in place of the non-labelled reagent previously employed.

The method of treatment or the compound for use in the treatment of cancer may involve, in addition to the NUC-1031 and the platinum agent, such as carboplatin, conventional surgery or radiotherapy or chemotherapy. Such chemotherapy may include the administration of one or more other active agents.

Thus, the, each or any one of the pharmaceutical formulations may comprise, or a patient may be treated with another active agent.

The one or more other active agents may be one or more of the following categories of anti-tumour agents:
(i) antiproliferative/antineoplastic drugs and combinations thereof, such as alkylating agents (for example cyclophosphamide, nitrogen mustard, bendamustin, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, pemetrexed, cytosine arabinoside, and hydroxyurea); antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); proteasome inhibitors, for example carfilzomib and bortezomib; interferon therapy; and topoisomerase inhibitors (for example epipodophyllotoxins like etoposide and teniposide, amsacrine, topotecan, mitoxantrone and camptothecin);
(ii) cytostatic agents such as antiestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5a-reductase such as finasteride;
(iii) anti-invasion agents, for example dasatinib and bosutinib (SKI-606), and metalloproteinase inhibitors, inhibitors of urokinase plasminogen activator receptor function or antibodies to Heparanase;
(iv) inhibitors of growth factor function: for example such inhibitors include growth factor antibodies and growth factor receptor antibodies, for example the anti-erbB2 antibody trastuzumab [HerceptinTM], the anti-EGFR antibody panitumumab, the anti-erbB1 antibody cetuximab, mirvetuximab soravtansine (IMGN853), tyrosine kinase inhibitors, for example inhibitors of the epidermal growth factor family (for example EGFR family tyrosine kinase inhibitors such as gefitinib, erlotinib and 6-acrylamido-N-(3-chloro-4-fluorophenyl)-7-(3-morpholinopropoxy)-quinazolin-4-amine (Cl 1033), erbB2 tyrosine kinase inhibitors such as lapatinib); inhibitors of the hepatocyte growth factor family; inhibitors of the insulin growth factor family; modulators of protein regulators of cell apoptosis (for example Bcl-2 inhibitors); inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib , tipifarnib and lonafarnib), inhibitors of cell signalling through MEK and/or AKT kinases, c-kit inhibitors, abl kinase inhibitors, P13 kinase inhibitors, Plt3 kinase inhibitors, CSF-1R kinase inhibitors, IGF receptor, kinase inhibitors; aurora kinase inhibitors and cyclin dependent kinase inhibitors such as CDK2 and/or CDK4 inhibitors;
(v) antiangiogenic agents such as those which inhibit the effects of vascular endothelial growth factor, [for example the anti-vascular endothelial cell growth factor antibody bevacizumab (AvastinTM); thalidomide; lenalidomide; and for example, a VEGF receptor tyrosine kinase inhibitor such as vandetanib, vatalanib, sunitinib, axitinib and pazopanib;
(vi) gene therapy approaches, including for example approaches to replace aberrant genes such as aberrant p53 or aberrant BRCA1 or BRCA2;
(vii) immunotherapy approaches, including for example antibody therapy such as alemtuzumab, rituximab, ibritumomab tiuxetan (Zevalin®) and ofatumumab; interferons such as interferon a; interleukins such as IL-2 (aldesleukin); interleukin inhibitors for example IRAK4 inhibitors; cancer vaccines including prophylactic and treatment vaccines such as HPV vaccines, for example Gardasil, Cervarix, Oncophage and Sipuleucel-T (Provenge); and toll-like receptor modulators for example TLR-7 or TLR-9 agonists; and (viii) cytotoxic agents for example fludaribine (fludara), cladribine, pentostatin (Nipent™);
(ix) steroids such as corticosteroids, including glucocorticoids and mineralocorticoids, for example aclometasone, aclometasone dipropionate, aldosterone, amcinonide, beclomethasone, beclomethasone dipropionate, betamethasone, betamethasone dipropionate, betamethasone sodium phosphate, betamethasone valerate, budesonide, clobetasone, clobetasone butyrate, clobetasol propionate, cloprednol, cortisone, cortisone acetate, cortivazol, deoxycortone, desonide, desoximetasone, dexamethasone, dexamethasone sodium phosphate, dexamethasone isonicotinate, difluorocortolone, fluclorolone, flumethasone, flunisolide, fluocinolone, fluocinolone acetonide, fluocinonide, fluocortin butyl, fluorocortisone, fluorocortolone, fluocortolone caproate, fluocortolone pivalate, fluorometholone, fluprednidene, fluprednidene acetate, flurandrenolone, fluticasone, fluticasone propionate, halcinonide, hydrocortisone, hydrocortisone acetate, hydrocortisone butyrate, hydrocortisone aceponate, hydrocortisone buteprate, hydrocortisone valerate, icomethasone, icomethasone enbutate, meprednisone, methylprednisolone, mometasone paramethasone, mometasone furoate monohydrate, prednicarbate, prednisolone, prednisone, tixocortol, tixocortol pivalate, triamcinolone, triamcinolone acetonide, triamcinolone alcohol and their respective pharmaceutically acceptable derivatives. A combination of steroids may be used, for example a combination of two or more steroids mentioned in this paragraph;
(x) targeted therapies, for example Pl3Kd inhibitors, for example idelalisib and perifosine; or immunotherapy compounds including anti-PD-1, anti- PD-L1 and anti-CTLA4 molecule, such as Nivolumab, pembrolizumab, pidilizumab, atezolizumab, durvalumab and avelumab; and CAR T cell therapies.

The one or more other active agents may also be antibiotics.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### EXAMPLES

### Example 1

### Clinical Study of Combination of NUC-1031 and carboplatin in Patients with Recurrent Ovarian Cancer

A Phase IB open label, dose-escalation study, PRO-002, was conducted to assess the safety, pharmacokinetics and clinical activity of NUC-1031 given on days 1 & 8 with carboplatin on day 1, q3-weekly for 6 cycles in participants with recurrent ovarian cancer.

The patients received NUC-1031 (Acelarin) administered as a slow bolus intravenous injection on days 1& 8 of a 21 day- cycle regimen alongside carboplatin on day 1 of a 21 day regimen. Each patient received up to 6 cycles of treatment.

NUC-1031 is presented as a single dose intravenous injection in a clear vial containing 250 mg/ml NUC-1031 in a solution of dimethylacetamide (DMA) and normal saline in the ratio of 80:20. The product is a clear yellow solution, free from visible particles

In the study, most patients were treated with the S-epimer of Acelerin. Several patients received a 2:1 mixture of the S:R epimers. There is not believed to be any significant pharmacological difference between the two epimers but it has been found that the S-epimer is easier to formulate.

The majority of platinum sensitive patients received a NUC-1031 dose of 500 mg/m² and a carboplatin dose of AUC 5.

Table 1 summarises the data obtained from the study with patients divided into those with platinum resistant cancers and those with platinum sensitive or partially sensitive cancers.

Table 1 also shows data obtained from the earlier study on gemcitabine and carboplatin in combination.

It is important to appreciate that even though the Acelarin (NUC-1031)+carboplatin patients were only treated for 4% months, significant beneficial effects with substantially reduced amounts of toxic side effects were seen in the patients. It would be predicted that longer durations of treatment would yield even better efficacy results.

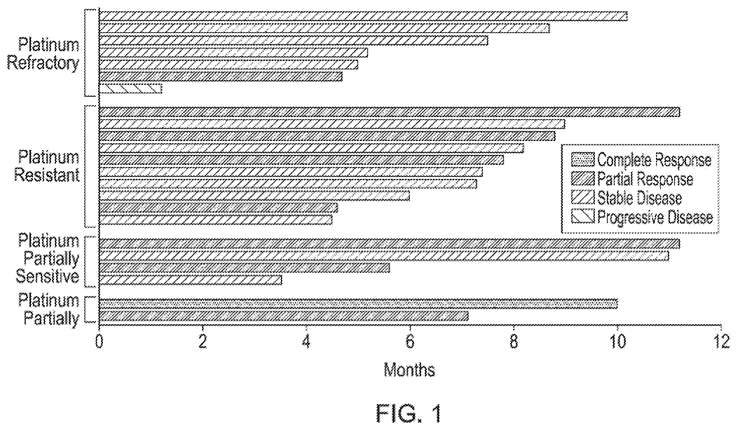

As a general rule of thumb, the PFS values are expected to halve for each additional line of therapy given. This a PFS of 8 months for second line patient group would be expected to translate to an approximate PFS of 4 months in third line and 2 months in 4th line. As can be seen from Table 1, although the media PFS for the NUC-1031/carboplatin combination was the same as that detected with gemcitabine/carboplatin (both 8.6 months), the patients treated with NUC-1031/carbo were third line, whereas those with gem/carbo were second line. Interestingly, patients that were resistant (resistant and refractory) treated in 4th line by NUC-1031/carboplatin had a PFS of 7.4 months. It is surprising to see such a relatively high PFS for fourth line patients. This demonstrates, that even in patients that have received 3 or more lines of prior therapy NUC-1031/carboplatin yields beneficial results. As can be seen in FIG. 1, most Pt refractory patients had stable disease with one partial responder and only one patient with progressive disease (bottom patient in refractory group).

FIG. 1 shows the PFS by platinum status. PFS 7.4; n=23; last dose given after approximately 4.5 months.

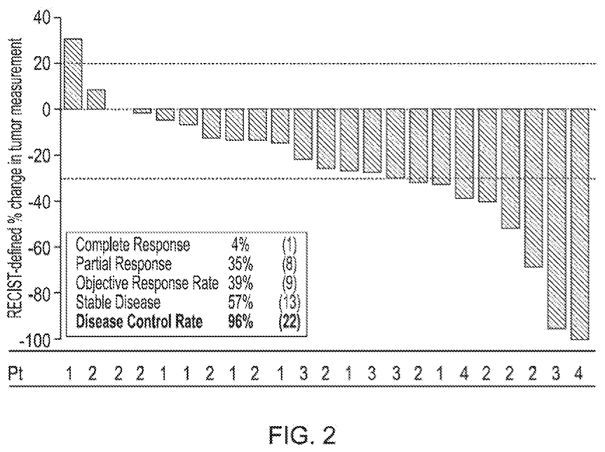

FIG. 2 shows a waterfall plot of percentage change in tumour measurements for each patient. The platinum status of each patient is shown beneath the plot. Key: Pt=Platinum status; 1=refractory (7), 2=resistant (10), 3=partially sensitive (4), 4=sensitive (2).

TABLE 1

| | Phase 1b Study | | | Gemzar Label |
|---|---|---|---|---|
| | Acelarin + | Acelarin + | Acelarin + | Gemcitabine + |
| | Carboplatin | Carboplatin | Carboplatin | Carboplatin |
| | (n = 23) | (n = 17) | (n = 6) | (n-178) |
| PRIOR STATUS | | | | |
| Resistance Status | Resistant & | Resistant | Sensitive | Sensitive |
| | Sensitive | | | |
| Line of Chemo (median) | 4^{th} Line | 4^{th} Line | 3^{rd} Line | 2^{nd} Line |
| RESPONSE | | | | |
| CR | 4% | — | 17% | 15% |
| PR | 35% | 29% | 50% | 33% |
| ORR | 39% | 29% | 67% | 47% |
| SD | 57% | 65% | 33% | 38% |
| DCR | 96% | 94% | 100% | 85% |
| PFS | 7.4 | 7.4 | 8.6 | 8.6 |
| | months | months | months | months |
| SAFETY (Grade 3-4) | | | | |
| Neutropenia | 52% | 53% | 50% | 71% |
| Thrombocytopenia | 30% | 41% | 0% | 35% |

Abbreviations:
CR—complete response;
PR—partial response;
ORR—objective response rate;
SD—stable disease;
DCR—disease control rate; and,
PFS—progression free survival

Of the 17 patients in the resistant group (e.g. see column 2), 10 were platinum resistant and 7 platinum refractory. Of the sensitive patients (e.g. see column 3), 2 were platinum sensitive and 4 platinum partially sensitive. The individual patients and their platinum status groupings is shown in FIG. 1. Only one patient in the trial (lowest refractory patient in FIG. 1) showed progressive disease.

1. 2′-Deoxy-2′,2′-difluoro-D-cytidine-5′-O-[phenyl (benzoxy- L-alaninyl)] phosphate) (NUC-1031), or a pharmaceutically acceptable salt or solvate thereof for use in a method of treating cancer in combination with a platinum agent, wherein the method comprises determining the platinum status of the patient's cancer and administering the NUC-1031 and platinum agent to a patient identified as likely to respond to treatment based on their platinum status.

2. NUC-1031 for use as claimed in claim 1, wherein number of previous treatment regimens the patient has received is also taken into consideration when identifying whether the patient is likely to respond to the treatment.

3. NUC-1031 for use as claimed in claim 2, wherein the patient is treated with the NUC-1031 and platinum agent if: (i) the patient's cancer is platinum sensitive or partially sensitive and the patient has received at least one prior treatment regime; (ii) the patient's cancer is platinum resistant and the patient has received at least two prior treatment regimes; or (iii) the patient's cancer is platinum refractory and the patient has received at least three prior treatment regimes.

4. NUC-1031 for the use as claimed in any of the preceding claims, wherein the platinum agent is carboplatin.

5. NUC-1031 for the use as claimed in any of the preceding claims, wherein the NUC-1031 is administered at a dose in the range from 350 to 750 mg/m² and the carboplatin is administered at a dose in the range from AUC 4 to AUC 6.

6. NUC-1031 for the use a claimed in claim 4, wherein the platinum agent is cisplatin.

7. NUC-1031 for the use as claimed in any of the preceding claims, wherein the cancer is selected from pancreatic cancer, lung cancer, bladder cancer, breast cancer, biliary cancer, colorectal cancer and a gynaecological cancer (e.g. a cancer selected from cancer of the uterus, cancer of the fallopian tube, endometrial cancer, ovarian cancer, peritoneal cancer and cervical cancer).

8. NUC-1031 for the use as claimed in claim 7, wherein the cancer is selected from ovarian cancer, fallopian tube cancer and peritoneal cancer.

9. NUC-1031 for the use as claimed in any of the preceding claims, wherein the NUC-1031 is administered at a starting dose of approximately 500 mg/m².

10. NUC-1031 for the use as claimed in any of claims 1, 2, 3, 4, 5, 7, 8 and 9, wherein the carboplatin is administered at a starting dose of approximately AUC5.

11. NUC-1031 for the use as claimed in any of claims 1, 2, 3, 4, 5, 7, 8, 9 and 10 wherein NUC-1031 is administered on day 1 and day 8 of a 21 day cycle and carboplatin is administered on day 1 of the 21 day cycle.

12. NUC-1031 for the use as claimed in any of the preceding claims, wherein treatment is provided to a subject in need thereof for at least 5 cycles of treatment.

13. NUC-1031 for the use as claimed in any of the preceding claims, wherein the NUC-1031 is gemcitabine-[phenyl-benzoxy-L-alaninyI)]-(S)-phosphate in substantially diastereomerically pure form.

14. NUC-1031 for the use as claimed in any of the preceding claims, wherein the NUC-1031 is a mixture of phosphate diastereoisomers.

15. NUC-1031 for the use as claimed in any of the preceding claims, wherein the NUC-1031 is in the form of the free base.

16. NUC-1031 for the use as claimed in any of the preceding claims, wherein the cancer is platinum sensitive.

17. A method of treating cancer, the method comprising administering to a subject in need thereof a therapeutically effective amount of gemcitabine-[phenyl-benzoxy-L-alaninyl)]-phosphate (NUC-1031), or a pharmaceutically acceptable salt or solvate thereof, in combination with carboplatin, wherein the NUC-1031 is administered at a dose in the range from 350 to 750 mg/m² and the carboplatin is administered at a dose in the range from AUC 4 to AUC 6, and the cancer is a platinum sensitive cancer or a platinum partially sensitive cancer.

18. A method of claim 17, wherein the cancer is selected from lung cancer, bladder cancer, breast cancer and a gynaecological cancer (e.g. a cancer selected from cancer of the uterus, cancer of the fallopian tube, endometrial cancer, ovarian cancer, peritoneal cancer and cervical cancer).

19. A method of claim 18, wherein the cancer is selected from ovarian cancer, fallopian tube cancer; and peritoneal cancer.

20. A method of any one of claims 17 to 19, wherein the NUC-1031 is administered at a dose of approximately 500 mg/m².

21. A method of any one of claims 17 to 20, wherein the carboplatin is administered at a dose of approximately AUC5.

22. A method of any one of claims 17 to 21, wherein NUC-1031 is administered on day 1 and day 8 of a 21 day cycle and carboplatin is administered on day 1 of the 21 day cycle.

23. A method of any one of claims 17 to 22, wherein treatment is provided to a subject in need thereof for at least 5 cycles of treatment.

24. A method of any one of claims 17 to 23, wherein the NUC-1031 is gemcitabine-[phenyl-benzoxy-L-alaninyl)]-(S)-phosphate in substantially diastereomerically pure form.

25. A method of any one of claims 17 to 23, wherein the NUC-1031 is a mixture of phosphate diastereoisomers.

26. A method of any one of claims 17 to 25, wherein the NUC-1031 is in the form of the free base.

27. A method of any one of claims 17 to 26, wherein the cancer is platinum sensitive.
